(19) United States
(12) Patent Application Publication
(10) Pub. No.: US 2010/0266716 A1
(43) Pub. Date: Oct. 21, 2010

(54) Natural Photodynamic Agents and their use

(76) Inventors: Merle E. Olson, Calgary (CA); Nicholas D. Allan, Calgary (CA)

Correspondence Address: WILLIAM J BUNDREN;THE LAW OFFICE OF WILLIAM J BUNDREN, 734 LaRue Road, Millersville, MD, 21108, US

(21) Appl. No.: 12/682,929

(22) PCT Filed: Oct. 27, 2008
(86) PCT No.: PCT/CA2008/001928

§ 371 Date: Jul. 6, 2010

Related U.S. Application Data
(60) Provisional application No. 60/982,419, filed on Oct. 25, 2007.

(51) Int. Cl. A61K 36/906; A61K 36/185; A01N 65/08; A01N 65/48; A61K 31/409; A01N 43/90; A01P 1/00; A01P 3/00; A01P 13/00; A01P 5/00; A61P 31/04; A61P 31/10; A61P 31/12; A61P 33/00

(52) U.S. Cl. 424/756; 424/725; 514/410

(57) ABSTRACT

The present invention is safe photodynamic agents and their use in treating microbial contamination.

This application is claims priority from U.S. Provisional Application No. 60/982,419 filed Oct. 25, 2007.

### I. FIELD OF THE INVENTION

This invention relates to compositions comprising natural photodynamic agents and methods for treating and preventing contamination.

This invention also relates to the use of photoactive natural products that can be safely applied to hard surfaces, food surfaces, animal and plant surfaces to prevent, treat, decontaminate or eliminate infections, contaminants, spoilage microorganisms and foodborne pathogens.

### II. BACKGROUND OF THE INVENTION

The photoactive agent that is used for therapeutic applications is a photosensitizer. Photosensitizers have shown great promise in cancer therapy. See, for example, Peng et al, Ultrastructural Pathology, 20:109 (1996), Reddi, J. Photochem. Photobiol. B: Biol., 37:189 (1997), Jori, J. Photochem. Photobiol. B: Biol., 36:87 (1996), Calzavara-Pinton et al, J. Photochem. Photobiol. B: Biol., 36:225 (1996), Geze et al, J. Photochem. Photobiol. B: Biol., 20:23 (1993) and Spikes, J. Photochem. Photobiol. B: Biol., 6:259 (1990), the disclosures of each of which are hereby incorporated by reference herein in their entirety. Upon application of the appropriate light, photosensitizers can photochemically (e.g., through photooxidation, photoreduction and the like) change into a form that is toxic to the cell or surrounding tissue. For example, following excitation of a photosensitizer to a long-lived excited singlet and/or triplet state, a targeted tumor is destroyed either by free radical products (a Type I mechanism) generated by quantum energy transfer or by the highly reactive singlet oxygen species (a Type II mechanism). Major biological target molecules of the singlet oxygen species and/or free radical products include nucleic acids, enzymes and cell membranes. A secondary therapeutic effect of the present methods involves the release of pathophysiologic products such as prostaglandins, thromboxanes and leukotrienes by tissue exposed to the effects of activated photosensitizers.

To date, only Photofrin® has been approved by health boards in Canada, Japan, the Netherlands, and the United States. In spite of the favorable results obtained with Photofrin®, some important factors still limit the efficacy of PDT, including its complex composition, the low extinction coefficient in the red spectral region and the prolonged cutaneous phototoxicity. Therefore, there is a need for more ideally suited photosensitizers.

The present invention addresses the growing need for safe and effective methods of treatment of contaminants, spoilage microorganisms, and human, animal, plant, and food borne pathogens in a wide range of industries.

### III. SUMMARY OF THE INVENTION

The present invention is the use of one or more food dyes or colors as a photodynamic agent (PDA), including in photodynamic therapy, to treat or remove microbial contamination. As used herein, food colors or dyes refers to natural, synthetic, Lake, and blended food colors.

It has been discovered that certain edible or ingestible food dyes are equal to or superior to synthetic chemical photodynamic agents. The advantages of such dyes are their non-toxic nature, their ability to be safely consumed, and their breakdown to safe and environmental friendly products. Such photoactive dyes are effective against pathogenic and spoilage microorganisms (bacteria, fungi, algae, viruses, protozoa).

This invention teaches a method to treat or decontaminate a surface, food product, infected animal or plant tissue through the use of a safe natural or synthetic food coloring agent that has photodynamic properties.

The present invention provides a composition for the treatment of infections and contaminants using one or more of the photodynamic agents of the present invention.

An advantage of the present invention is that it employs non-toxic agents, all of which are known to be safe for use and/or consumption.

### IV. DETAILED DESCRIPTION OF THE INVENTION

The present invention comprises a composition comprising one or more natural or synthetic photoactive agents, typically one or more food dyes or colors. The present invention also involves using these photoactive agents to treat contamination or infection caused by microorganisms.

Some embodiments of the invention include contacting the microorganism with a composition comprising one or more natural or synthetic photoactive agents, and exposing the photoactive agent to a light source appropriate to activate the photoactive agent. In accordance with the present invention, contacting a microorganism with an activated photoactive agent results in microorganism apoptosis.

In preferred embodiments of the invention, the photoactive agents are selected from natural, synthetic, Lake, or blended food dyes or colors. In the most preferred embodiments of the invention, food dyes include one or more natural photoactive agents selected from the group consisting of annatto extract (contains the carotenoids bixin and norbixin), turmeric, heme, and porphyrin.

The present invention comprises administering at least one photodynamic agent(s) to one or more surfaces, activating said photodynamic agent by exposing the surface and/or microorganism to photoradiation, thereby inactivating one or more organisms or microorganisms.

Some embodiments of the invention include contacting or treating a smooth surface with a PDA of the present invention. In these embodiments of the invention, it has been found that treating smooth surfaces sometimes provides better results, due in part to ease with which light may contact the surface. That is, rough surfaces are sometimes more difficult to expose light sufficiently to decontaminate the surface.

In one embodiment of the present invention, the photodynamic agents are effective in killing microorganisms in the planktonic and biofilm state.

Upon irradiation with light, the agents of the present invention can generate active oxygen species, including singlet oxygen (¹O₂) superoxide anion radical (O₂⁻), and hydroxyl radical (HO⁻), and possesses high photodynamic activity.

Photodynamic agents of the present invention may include compounds which preferentially adsorb to nucleic acids, thus focusing their photodynamic effect upon microorganisms and viruses with little or no effect upon accompanying cells or proteins.

Another embodiment of the present invention comprises photodynamic agents that provide treatment or elimination of planktonic and biofilm microorganism from surfaces including, but not limited to living and non-living hard surfaces, food surfaces and living tissue surfaces.

In a further embodiment of the present invention the organism and microorganisms are viral, bacterial, fungal, algal or parasitic. Microorganisms include viruses (both extracellular and intracellular), bacteria, bacteriophages, fungi, algae, parasites (nematode, cestode, trematode, arthropod), and insects.

Examples of microorganisms include but are not limited to Pseudomonas, E. coli, Campylobacter, Salmonella, Listeria, Staphylococcus, Aspergillus, and Fusarium.

The present invention also comprises a method of treating a disease, infection or contamination by administering a sufficient amount of at least one PDA agent of the present invention, and photoactivating the agent(s). Typically, the PDA agent may be activated, using known procedures, e.g., by exposing the derivative to a pre-determined wavelength of light, including natural sunlight.

The present invention also comprises a method of photodynamic treatment comprising administering a composition comprising one or more PDA agents of the present invention.

In the present invention photoradiation may comprise light in the visible spectrum, the ultraviolet spectrum, or light in both the visible and ultraviolet spectra. Any suitable wavelength or wavelengths of light may be used in any proportion and energy that produces the desired level of inactivation of microorganisms.

The surface that has been treated with the natural photodynamic agent is exposed to photoradiation of the appropriate wavelength to activate the photosensitizer, using an amount of photoradiation sufficient to activate the photosensitizer as described herein, but less than that which would cause non-specific damage to the hard surface, meat, vegetable produce or living biological tissue. The wavelength used will depend on the photosensitizer selected and composition of the surface. Nonspecific damage is damage that damages all components.

In the present invention, the photoradiation in both the ultraviolet and visible spectra may be supplied concurrently or sequentially, with the visible portion preferably being supplied first. The photoradiation source may be a simple lamp or may consist of multiple lamps radiating at differing wavelengths. The photoradiation source should be capable of delivering a sufficient amount of light to activate the photosensitizer.

In the present invention materials which may be treated include any surface where contamination may be found. Surface, as used herein, is intended to be broadly defined as including any place that can be exposed to light. It is intended to exclude in vivo applications, except where surgery might expose a surface. The following provides examples of such uses, a list that is not intended to limit the scope of the invention.

The photoactive agents of the present invention may be used on any surface where microbial contamination is an issue. Exemplary surfaces include, but are not limited to non-living materials such as hard surfaces (steel, plastic, glass, wood); a hard surface sanitizer; a hard surface disinfectant; a cold surface sanitizer (e.g., refrigerator, cold room, freezer); industrial biofouling inhibition and/or treatment (e.g., pulp and paper mills, mine tailings, oil/fuel/gas pipelines, milk transport lines, food transport lines, fuel lines, aviation fuel lines); medical devices; medical device coatings; food storage containers (including covers, treatment, or impregnated into the container material); Pule storage containers (including covers, treatment, or impregnated into the container material); contact lense compositions, including sanitizers and disinfectants; anti-corrosion agents, including but not limited to microbial induced corrosion;

The photoactive agents of the present invention may be used on any food or plant, as an additive or as a sanitizer, disinfectant, or preservative. Exemplary uses include on meat (e.g., beef, pork, and poultry; processed meats; hot dogs, manufactured foods), vegetables (e.g., lettuce, sprouts, and potatoes), fruit (e.g., apples, pears, and peaches); living plants (vegetables, fruits, ornamentals, seeds, flowers, cereal crops, oil crops, trees, bushes, shrubs); as a foliar spray (e.g., anti-bacterial, anti-parasitic, anti-aphid, anti-locust, anti-fungal sanitizer, food sanitizer, disinfectant); as a seed treatment (e.g., anti-bacterial, anti-parasitic, anti-aphid, anti-locust, anti-fungal sanitizer, food sanitizer, disinfectant); as a food additive to extend the life of processed and non-processed foods (including extending the storage life).

The photoactive agents of the present invention may be used on human and animal tissues (e.g., skin, eyes, mucus membranes); human, animal, or plant wound treatment; human or animal mucosal infection treatment; nasal MSRA treatments; human or animal ocular infection treatments, alone or in combination with eye drops;

The photoactive agents of the present invention may be used as an additive to any other composition where it might be useful to include a disinfectant, sanitizer, or the like. Examples include but are not limited to cosmetics (e.g., as a preservative or additive), including for anti-microbial or anti-aging cosmetics; a paint or ink additive (e.g., a hard surface sanitizer); in fabrics or textiles (e.g., consumer products such as furniture, clothing, sprouting wear and equipment); as an odor preventative or treatment; for textile detergent additives; for pet litter (e.g., to prevent or treat odors and/or as an anti-toxiplasma agent); as a waste water additive; as a grey water additive; as a potable water additive; as a soap additive; in a shampoo composition; in any detergent composition; a paint or stain additive (e.g., to prevent staining).

The photoactive agents of the present invention may be used in human, animal, or plant wound treatment compositions and products, including but not limited to bandages, sutures, ointments or compositions.

The photoactive agents of the present invention may be used for peptic ulcer treatment systems (e.g., H. pylori induced). The photoactive agents of the present invention are particularly useful in any organic, green, environmentally friendly, natural, or natural based composition.

The photoactive agents of the present invention may also be used in combination with, or included in a kit for, a light system or source.

A kit in accordance with the present invention may include one or more food colors or dyes; one or media for dissolving the color; one or more media for applying, contacting, spraying, or distributing the color; one or more light systems or sources; and combinations of any of the above.

The photoactive agents of the present invention are preferably used as a hard surface cleaner, disinfectant, or sanitizer; as a food cleaner, disinfectant, or sanitizer; or as a seed or foliar cleaner, disinfectant, or sanitizer.

In a further embodiment of the present invention the treatment is particularly useful in greenhouses with natural or artificial lighting. The technology can be used to treat viral, fungal, bacterial and parasitic plant diseases which affect leaves or the seed/fruit itself. The advantage is the plant or seed can be safely consumed following application of the photodynamic agent.

The photoactive agents are used in the compositions of the present invention in any amount sufficient to treat microorganisms and/or to treat or prevent contamination. Typically, the appropriate amount or concentration is the amount needed to generate sufficient singlet oxygen to affect the microorganism. One skilled in the art will recognize that the appropriate concentration for a particular photodynamic agent will depend in part on the type of light being used to activate the agent, composition's ingredients, the particular microorganism being treated, the structure of the microorganism (e.g., biofilm or planktonic, among others), and/or the type of delivery (e.g., spray or immersion, among other). Further, the examples suggest that some of the photodynamic agents are effective only within a concentration range. One skilled in the art may determine the range for a particular photodynamic agent by using conventional assays to determine the concentration where the activated agent has little effect on the microorganism (i.e., the low end of the concentration range) and by using conventional assays to determine the concentration where the amount of photodynamic agents inhibits the light source from activating the agent.

Generally, a concentration range from about 0.01% to about 80%, by weight, is suitable for most photodynamic agents under most conditions. The preferred concentration range is from about 0.1% to about 10% by weight, more preferably about 0.01% to about 1% by weight. It is intended however that the invention should not be limited by the concentration of the photodynamic agent in the composition.

One skilled in the art can determine other effective combinations of light, photodynamic agent, and concentration by conducting experiments known in the art, including but not limited to high through put screening, MIC testing, MBEC testing, bioassays, dose titration experiments (manual or robotic), computer modeling. Exemplary assays are shown in the examples. The particular assays described in the examples and the patents cited therein are particularly useful because they are all high throughput assays. Therefore, a significant number of possible photodynamic agents may be determined for both effectiveness and concentration in a short amount of time using only simple experiments.

In accordance with the present invention, a composition comprising annatto, annatto extract, norbixin, annatto powder, or annatto derivatives may be used in a concentration range from about 0.01% to about 80%, by weight, preferably between about 0.01% and about 15%, most preferably between about 1% and about 10%. For example, the examples show that annatto extract is useful against Staph A at concentrations greater than about 1% using either LED light or fluorescent light; and against Salmonella at concentrations greater than about 0.01% using either LED light or fluorescent light.

In accordance with the present invention, a composition comprising heme (hemin and/or hemoglobin) may be used in a concentration range from about 0.01% to about 80%, by weight, preferably between about 0.01% and about 15%, most preferably between about 1% and about 10%. For example, the examples show that hemin and hemoglobin is useful against Salmonella at concentrations greater than about 0.01% using fluorescent light or greater than about 1% using LED light.

In accordance with the present invention, a composition comprising turmeric may be used in a concentration range from about 0.01% to about 80%, by weight, preferably between about 0.01% and about 15%, most preferably between about 1% and about 10%. For example, the examples show that turmeric against Salmonella at concentrations greater than about 0.01% using either fluorescent light and greater than about 1% using LED light; and against Staph A using greater than about 0.1% using fluorescent light.

In accordance with the present invention, a composition comprising porphyrin may be used in a concentration range from about 0.01% to about 80%, by weight, preferably between about 0.01% and about 15%, most preferably between about 1% and about 10%. For example, the examples show that porphyrin against Salmonella at concentrations greater than about 0.01% using either fluorescent light and greater than about 1% using LED light; and against Staph A using greater than about 0.1% using fluorescent light.

One skilled in the art can determine other effective combinations (e.g., of light, of food dye, and/or of food dye concentration) by conducting experiments known in the art. Exemplary tests include but are not limited to high throughput screening, MIC testing, MBEC testing, bioassays, dose titration experiments (manual or robotic), and computer modeling.

The compositions of the present invention may be prepared in various formulations depending on intended use and/or delivery mechanism, e.g., for topical or cutaneous administration. Topical or cutaneous delivery may also include aerosol formulations. The composition may be water based or oil based; and/or may be in the form of a liquid, gel, cream, ointment, lotion, or oil.

The compositions may also contain conventional pharmaceutical carriers and excipients appropriate for the type of administration contemplated. Other agents which may be included are stabilizing agents and skin penetration enhancing agents. Such solutions also may contain pharmaceutically acceptable buffers, emulsifiers, surfactants, and electrolytes such as sodium chloride. Acceptable carriers include aqueous buffer solutions, e.g., tris(hydroxymethyl)aminomethane and its salts, phosphate, citrate, bicarbonate, and the like, sterile water for injection, physiological saline, and balanced ionic solutions containing chloride and/or bicarbonate salts of normal blood plasma cations such as Ca²⁺, Na⁺, K⁺ and Mg²⁺. The carriers may contain a chelating agent, e.g., ethylenediaminetetraacetic acid, calcium disodium salt, or other pharmaceutically acceptable chelating agents. Compositions for oral administration may also contain flavoring agents and other ingredients for enhancing their organoleptic qualities. Formulations for topical delivery may also contain liquid or semisolid excipients to assist in the penetration or aerosol spray of the photoactive molecule.

The compositions and stabilizing materials of the present invention may also comprise or be used in combination with an additional bioactive agent. Suitable bioactive agents include, for example, antineoplastic agents, blood products, biological response modifiers, anti-fungal agents, hormones, steroids, vitamins, peptides, peptide analogs, enzymes, anti-allergenic agents, anti-coagulation agents, circulatory agents, anti-tubercular agents, anti-viral agents, anti-anginal agents, antibiotics, anti-inflammatory agents, analgesics, anti-protozoan agents, anti-rheumatic agents, narcotics, cardiac glycoside agents, chelates, neuromuscular blocking agents, sedatives (hypnotics), local anesthetic agents, general anesthetic agents, radioactive particles, radioactive ions, X-ray contrast agents, monoclonal antibodies, polyclonal antibodies and genetic material.

The compositions of the present invention may also include one or more food preservation agents, including but not limited to sodium benzoate and benzoic acid; calcium, sodium propionate, and propionic acid; calcium, potassium, sodium sorbate, sorbic acid; sodium and potassium sulfite. calcium, sodium ascorbate, and ascorbic acid; butylated hydroxyanisole (BHA) and butylated hydroxytoluene (BHT); lecithin; and sodium and potassium sulfite and sulfur dioxide. ethylenediamine-tetraacetic acid (EDTA), citric acid, sorbitol, and tartaric acid.

As one skilled in the art will recognize, any of the stabilizing materials and/or vesicle compositions may be lyophilized for storage, and reconstituted or rehydrated, for example, with an aqueous medium (such as sterile water, phosphate buffered solution, or aqueous saline solution), with the aid of vigorous agitation. Lyophilized preparations generally have the advantage of greater shelf life. To prevent agglutination or fusion of the lipids and/or vesicles as a result of lyophilization, it may be useful to include additives which prevent such fusion or agglutination from occurring. Additives which may be useful include sorbitol, mannitol, sodium chloride, glucose, dextrose, trehalose, polyvinyl-pyrrolidone and poly(ethylene glycol) (PEG), for example, PEG 400. These and other additives are described in the literature, such as in the U.S. Pharmacopeia, USP XXII, NF XVII, The United States Pharmacopeia, The National Formulary, United States Pharmacopeial Convention Inc., 12601 Twinbrook Parkway, Rockville, Md. 20852, the disclosure of which is hereby incorporated herein by reference in its entirety.

For topical applications, the stabilizing materials and/or vesicles may be used alone, may be mixed with one or more solubilizing agents, such as dimethylsulfoxide (DMSO), or may be used with a delivery vehicle, and applied to the skin or mucosal membranes. Penetrating and/or solubilizing agents useful for the topical application of the stabilizing materials and/or vesicles are well known in the art. Stabilizing materials and/or vesicles formulated with penetration enhancing agents may be administered transdermally in a patch or reservoir with a permeable membrane applied to the skin. The use of rupturing ultrasound may increase transdermal delivery of photoactive agents. Further, a mechanism may be used to monitor and modulate delivery of the photoactive agents. For example, ultrasound may be used to visually monitor the bursting of the gas filled vesicles and modulate delivery of the photoactive agents and/or bioactive agents and/or a hydrophone may be used to detect the sound of the bursting of the gas filled vesicles and modulate delivery of the photoactive agents and/or bioactive agents.

Generally, the compositions of the invention are administered in the form of an aqueous suspension such as in water or a saline solution (e.g., phosphate buffered saline). The water may be sterile but is preferably not sterile. Also, preferably the saline solution is an isotonic saline solution, although, if desired, the saline solution may be hypotonic (e.g., about 0.3 to about 0.5% NaCl). The solution may be buffered, if desired, to provide a pH range of about 5 to about 7.4. Preferably, dextrose or glucose is included in the media. Other solutions that may be used for administration of the compositions of the present invention include oils, such as, for example, almond oil, corn oil, cottonseed oil, ethyl oleate, isopropyl myristate, isopropyl palmitate, mineral oil, myristyl alcohol, octyldodecanol, olive oil, peanut oil, persic oil, sesame oil, soybean oil, squalene and fluorinated oils. Suitable fluorinated oils are described in U.S. Pat. No. 5,344,930, the disclosure of which is hereby incorporated by reference herein in its entirety.

The PDAs and/or the compositions of the present invention may also include one or nutrients for growing the microorganism, including planktonic or biofilm microorganisms. Exemplary nutrients include but are not limited to a nutrient broth such as Mueller Hinton Broth and those described in PCT/CA2006/001226, and U.S. Pat. Nos. 7,041,470; 6,410,256; 6,326,190; 6,051,423; and 6,599,714, all incorporated herein by reference in their entirety.

In some embodiments, the photoactive agents may be covalently bonded or conjugated to the stabilizing materials described herein, including lipids, polymers, proteins and surfactants, preferably lipids or surfactants, such as lipid conjugated rhodamine, lipid conjugated fluorescein, lipid conjugated N-(lissamine-rhodamine-.beta.-sulfonyl), and those described by MacDonald, J. Biol. Chem., 265(23): 13533-9 (1990), Leenhouts et al, Biochim. Biophys. Acta, 1237(2):121-6 (1995), Ahlers et al, Biophys. J., 63(3):823-38 (1992) and Ebato et al, Anal. Chem., 66(10):1683-9 (1994), the disclosures of which are hereby incorporated by reference herein in their entirety. Alternatively, where a —OH group or —COO⁻. group is at the terminus of the photoactive agent, the photoactive agent may esterify with a lipid.

The photodynamic therapy agents of the present invention may also be conjugated to binding agents that bind pre-determined cells or structures in vitro or in vivo. Conjugation with monoclonal antibodies (e.g., immunoconjugates) affords specificity with respect to the treatment of a variety of diseases and conditions.

As used herein, photodynamic agents or photoactive agents, or similar terms, are defined as any compound that absorbs radiation of one or more defined wavelengths and subsequently utilizes the absorbed energy to carry out a chemical process. These agents include but are not limited to any compound or material that is active in light or that responds to light, including, for example, chromophores (e.g., materials that absorb light at a given wavelength), fluorophores (e.g., materials that emit light at a given wavelength), photosensitizers (e.g., materials that can cause necrosis of tissue and/or cell death in vitro and/or in vivo), fluorescent materials, phosphorescent materials and the like, that may be used in diagnostic or therapeutic applications. “Light” refers to all sources of light including the ultraviolet (UV) region, the visible region and/or the infrared (IR) region of the spectrum.

In the present invention, the photodynamic agents are one or more food dyes or colors. The preferred food dyes or colors are well known to those skilled in the art, and include natural, synthetic, Lake, and blended food colors, and combinations thereof. In general these colors may be selected from the group consisting of chlorophylls, carotenoids, flavenoids, quinonoids, coumarins, indigoids, curcuminoids, befalins, acthocyanins, cyanines, indocyanines, phthalocyanines, rhodamines, phenoxazines, phenothiazines, phenoselenazines, fluoresceins, porphyrins, benzoporphyrins, squaraines, corrins, croconiums, azo compounds, methine dyes, and indolenium. Natural food dyes include but are not limited to annatto extract, anthocyanins, B-carotene, beta APO 8 Carotenal, black currant, burnt sugar, Canthaxanthin, caramel, carbo medicinalis, carmine, carmine blue, carminic acid, carrot, chlorophyll, chlorophyllin, cochineal extract, copper chlorophyll, copper chlorophyllin, curcumin, curcumin/CU-chloro, grape, hibiscus, lutein, mixed carotenoids, paprika, riboflavin, spinach, stinging nettle, titanium dioxide, turmeric, aronia/red fruit, beet juice, paprika extract, and paprika oleoresin. Synthetic food colors include but are not limited to allura red, amaranth, black PN, carmoisine, fast red E, erythrosine, green S, patent blue V, ponceau 4R, quinoline yellow, Red 2G, sunset yellow, and tartrazine. Examples of Lake food colors include but are not limited to Lake allura red, Lake amaranth, Lake brillian blue FCF, Lake carmosine, Lake erythrosine, Lake indigo carmine, Lake ponceau 4R, Lake quinoline yellow, Lake sunset yellow, and Lake tartrazine.

As noted above, the photodynamic agent may be natural or synthetic; “natural” as used herein refers to existing in or formed by nature. “Synthetic”, as used herein, refers to artificial or formed through a chemical process by human agency. The PDS may also be any coloring agent derived from any of the above (e.g., annatto extract can be used to produce bixin (an oil-soluble apocarotenoid) or norbixin (a water-soluble derivative).

As used herein, the term “inactivation of a microorganism” means totally or partially preventing the microorganism from replicating, either by killing the microorganism or otherwise interfering with its ability to reproduce or function.

“Delivery vehicle” or “vehicle” refers to a composition, substance or material that can transport or carry in vivo or in vitro a photoactive agent, a bioactive agent and/or a targeting ligand. Suitable delivery vehicles include, for example, stabilizing materials, vesicles, liposomes, micelles, aerogels, clathrates, gas and/or gaseous precursor filled vesicles, emulsions, suspensions, dispersions, hexagonal H II phase structures, cochleates and the like.

“Stabilizing material” or “stabilizing compound” refers to any material which can improve the stability of compositions containing the photoactive agents, gases, gaseous precursors, liquids, bioactive agents and/or targeting ligands described herein, including, for example, mixtures, suspensions, emulsions, dispersions, vesicles, or the like. The improved stability involves, for example, the maintenance of a relatively balanced condition, and may be exemplified by increased resistance of the composition against destruction, decomposition, degradation, and the like. In the case of preferred embodiments involving vesicles filled with photoactive agents, gases, gaseous precursors, liquids, bioactive agents and/or targeting ligands, the stabilizing compounds may serve to either form the vesicles or stabilize the vesicles, in either way serving to minimize or substantially (including completely) prevent the escape of photoactive agents, gases, gaseous precursors, bioactive agents and/or targeting ligands from the vesicles until release is desired. The term “substantially” as used in the context of preventing escape of photoactive agents, gases, gaseous precursors, bioactive agents and/or targeting ligands from the vesicles means greater than about 50% is maintained entrapped in the vesicles until release is desired, preferably greater than about 60%, about 70%, about 80% or about 85% is maintained entrapped in the vesicles until release is desired, with about 90% being even more preferred. In particularly preferred embodiments, greater than about 95%, about 99% or about 100% of the photoactive agents, gases, gaseous precursors, bioactive agents and/or targeting ligands are maintained entrapped until release is desired. Exemplary stabilizing materials include lipids, proteins, polymers, carbohydrates and surfactants. The resulting mixture, suspension, emulsion or the like may comprise walls (e.g., films, membranes and the like) around the photoactive agent, targeting ligand, bioactive agent, gases and/or gaseous precursors, or may be substantially devoid of walls or membranes, if desired. The stabilizing may, if desired, form droplets. The stabilizing material may also comprise salts and/or sugars. In other embodiments, the stabilizing materials may be substantially (including completely) crosslinked. The stabilizing material may be neutral, positively or negatively charged.

As used herein, administering refers to any action that results in exposing or contacting one or more PDA agents of the present invention with a pre-determined cell, cells, or tissue, typically mammalian. As used herein, administering may be conducted in vivo, in vitro, or ex vivo. For example, a composition may be administered by spraying or washing. Administering also includes the direct application to cells of a composition according to the present invention.

As used herein, physiologically acceptable fluid refers to any fluid or additive suitable for combination with a composition containing a PDA agent. Typically these fluids are used as a diluent or carrier. Exemplary physiologically acceptable fluids include but are not limited to preservative solutions, saline solution, an isotonic (about 0.9%) saline solution, or about a 5% albumin solution or suspension. It is intended that the present invention is not to be limited by the type of physiologically acceptable fluid used. The composition may also include pharmaceutically acceptable carriers. Pharmaceutically accepted carriers include but are not limited to saline, sterile water, phosphate buffered saline, and the like. Other buffering agents, dispersing agents, and inert non-toxic substances suitable for delivery to an inert, dead, or living surface may be included in the compositions of the present invention. The compositions may be solutions, suspensions or any appropriate formulation suitable for administration, and are typically sterile and free of undesirable particulate matter. The compositions may be sterilized by conventional sterilization techniques.

The compounds of the present invention may also be used in conjunction with and conjugated to a number of other compounds, signaling agents, enhancers, and/or targeting agents.

Suitable enhancers include but are not limited to pKa modifiers, hypoxic cell radiosensitizers, and bioreductively activated anti-neoplastic agents, such as mitomycin C (preferably to effect or potentiate the toxicity of the compound in hypoxic cells or microorganisms). Suitable signaling agents include but are not limited to markers of apoptotic cell death or necrotic cell death, or regulatory molecules endogenous to cell cycle control or delay, preferably to potentiate the phototoxicity of the compound(s) by induction of apoptotic or necrotic cell death, or by inhibition of the repair of any form of lethal or potentially lethal damage (PLD).

As used herein, anti-biofilm agent refers to any element, chemical, biochemical, or the like that is effective against a biofilm. Typical anti-biofilm agents are those that have anti-microbial, anti-bacterial, anti-fungal or anti-algal properties. While the invention has been described in some detail by way of illustration and example, it should be understood that the invention is susceptible to various modifications and alternative forms, and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

### EXAMPLES

Microbial growth and evaluation procedures are based on procedures described in PCT/CA2006/001226, incorporated herein by reference in its entirety.

### Example 1

### Testing of Photocatalytic Activity of Food Dyes

Test microorganisms included Salmonella choleraesuis, Staphylococcus aureus, foodborne pathogens of humans. Test Compounds included Methylene blue, Hemoglobin (blood product), Hemin (blood product), Protoporphyrin (blood product), Annatto Extract (15% Norbixin), beet juice, Canthaxanthin (10%), Beta-carotene (1%), Purple carmine 50%, Carmine lake 60%, Black Carrot extract, Tomato lycopene (5%), sodium copper chlorophyllin, grape skin extract, paprika oleoresin, riboflavin, saffron, titanium dioxide, FD&C blue No1., turmeric oleoresin, FD&C blue No. 2, FD&C Green No. 3, FD&C Red No. 2 (Amaranth), FD&C Red No. 40, FD&C Yellow No. 5, and FD&C Yellow No. 6. Compounds were prepared as 1%, 0.1%, 0.01%, by weight, dissolved in water.

The light source was a full spectrum fluorescent light or LED light and the contact time was 1 and 16 hours. The study temperature was 37° C.

Approximately 10⁶ organisms in 150 uL Cation Adjusted Mueller Hinton Broth (CAMHB) were placed in each of the wells of a 96 peg MBEC device. The lid of the 96 peg MBEC device was placed on the bottom plate containing organism. The device was placed on a shaker, set to 110 rpm, and incubated at 37° C. for 24 hours to produce biofilms.

The challenge plates were set up using a sterile 96-well microtitre plate using aseptic techniques in the following manner (one organism per plate, per light exposure time, per light source). Test solutions (200 uL) were placed into each well. The lid was transferred to the challenge plate and exposed to the various light sources for the prescribed exposure times (16 hours). Following the treatment, the pegs were transferred to the recovery media (“recovery plate”) then wrapped in tin foil and incubated at 37° C. for 18 to 24 hours. The recovery plate(s) was wrapped in tin foil, incubated at 37° C. for 24 hours and read visually and by a plate reader, set to an optical density measurement of 630 nm, to determine Minimum Biofilm Eradication Concentration (MBEC) by looking at growth or no growth.

Both natural and artificial food dyes were effective as photodynamic agents in killing Salmonella choleraesuis and Staphylococcus aureus as biofilm organisms (Table 1). Effective food dyes included: Hemoglobin, Hemin, Protoporphyrin, Annatto extract, beet juice, purple carmine, carmine lake 6, black carrot extract, Grape skin, FD&C blue No1, and turmeric oleoresin. Most anti-microbial activity occurred after 16 hours of light exposure.

TABLE 1
Killing of Salmonella choleraesuis and Staphylococcus aureus
by different photodynamic agents and broad spectrum LED or
Fluorescent lights (16 Hours exposure)

| | Salmonella | | | | | |
|---|---|---|---|---|---|---|
| | cholerasuis | | | Staph aureus | | |
| | 1% | 0.1% | 0.01% | 1% | 0.1% | 0.01% |
| | Fluorescent Light | | | | | |
| Methylene blue | − | − | − | − | − | − |
| Hemoglobin | − | − | − | + | + | + |
| Hemin | − | − | − | + | + | + |
| Protoporphyrin | − | − | − | + | + | + |
| Annatto extract | − | − | − | − | + | + |
| Beet Juice | − | − | + | + | + | + |
| Canthaxanthin | + | + | + | + | + | + |
| β-Carotene | + | + | + | + | + | + |
| Purple Carmine | − | + | + | + | + | + |
| Carmine Lake 6 | − | − | − | + | + | + |
| Black Carrot | − | − | − | + | + | + |
| Tomato | + | + | + | + | + | + |
| chlorophyllin | + | + | + | + | + | + |
| Grape Skin | − | + | + | + | + | + |
| Paprika | + | + | + | + | + | + |
| Riboflavin | + | + | + | + | + | + |
| Saffron | + | + | + | + | + | + |
| TiO₂ | + | + | + | + | + | + |
| Blue No. 1 | − | − | − | + | + | + |
| Turmeric | − | − | − | − | − | + |
| Blue No. 2 | + | + | + | + | + | + |
| Green No. 3 | + | + | + | + | + | + |
| Red No. 2 | + | + | + | + | + | + |
| Red No. 40 | + | + | + | + | + | + |
| Yellow No. 5 | + | + | + | + | + | + |
| Yellow No. 6 | + | + | + | + | + | + |
| | LED Light | | | | | |
| Methylene blue | − | − | − | − | − | − |
| Hemoglobin | + | + | + | + | + | + |
| Hemin | − | + | + | + | + | + |
| Protoporphyrin | − | + | + | + | + | + |
| Annatto extract | − | − | − | − | + | + |
| Beet Juice | + | + | + | + | + | + |
| Canthaxanthin | + | + | + | + | + | + |
| β-Carotene | + | + | + | + | + | + |
| Purple Carmine | + | + | + | + | + | + |
| Carmine Lake 6 | − | + | + | + | + | + |
| Black Carrot | + | + | + | + | + | + |
| Tomato | + | + | + | + | + | + |
| chlorophyllin | + | + | + | + | + | + |
| Grape Skin | − | + | + | + | + | + |
| Paprika | + | + | + | + | + | + |
| Riboflavin | + | + | + | + | + | + |
| Saffron | + | + | + | + | + | + |
| TiO₂ | + | + | + | + | + | + |
| Blue No. 1 | + | + | + | + | + | + |
| Turmeric | − | + | + | + | + | + |
| Blue No. 2 | + | + | + | + | + | + |
| Green No. 3 | + | + | + | + | + | + |
| Red No. 2 | + | + | + | + | + | + |
| Red No. 40 | + | + | + | + | + | + |
| Yellow No. 5 | + | + | + | + | + | + |
| Yellow No. 6 | + | + | + | + | + | + |

“−” = biofilm eradication at the mentioned conditions (concentration, exposure time and Light source)
“+” = biofilm growth

Some agents, e.g., beet juice and purple carmine, are effective under these conditions at higher concentrations, but are shown to be effective against gram negative microorganisms.

### Example 2

### Testing of Photocatalytic Food Dyes Log Reduction

The test was conducted as shown in Example 1, but using Hemin (blood product), Protoporphyrin (blood product), Annatto Extract, and Turmeric. The group of tests organism also included E. Coli 0157H7.

All four photocatalytic dyes tested were effective anti-microbial agent, eliminating or reducing growth at all tested concentrations. Both broad spectrum light sources were effective but the fluorescent light provided superior antimicrobial activity when combined with the dyes.

TABLE 2
Average Log₁₀ counts of Salmonella choleraesuis, E. coli
0157H7, Staphylococcus aureus in 1%, 0.1% and 0.01% concentrations
of various photocatalytic agents when exposed to 16 hours of LED or
Fluorescent light. Samples were run in triplicate.

| | LED Light | | | Fluorescent Light | | |
|---|---|---|---|---|---|---|
| | 1% | 0.1% | 0.01% | 1% | 0.1% | 0.01% |
| E coli | | | | | | |
| Methylene blue (positive | 0.00 | 0.00 | 1.77 | 0.35 | 0.79 | 3.41 |
| control) | | | | | | |
| Hemin | 0.00 | 4.22 | 4.18 | 3.06 | 3.79 | 3.17 |
| Protoporphyrin | 3.60 | 3.48 | 4.04 | 0.00 | 0.57 | 0.44 |
| Annatto Extract | 0.00 | 4.04 | 4.45 | 0.00 | 0.00 | 1.34 |
| Turmeric | 4.61 | 3.26 | 2.91 | 0.00 | 0.73 | 2.05 |
| Control | 6.36 | 6.36 | 6.36 | 6.51 | 6.51 | 6.51 |
| Staph. aureus | | | | | | |
| Methylene blue (positive | 4.84 | 5.40 | 5.71 | 0.00 | 0.00 | 3.20 |
| control) | | | | | | |
| Hemin | 1.51 | 3.75 | 5.52 | 0.00 | 0.79 | 2.21 |
| Protoporphyrin | 3.10 | 0.50 | 1.29 | 0.00 | 0.00 | 0.00 |
| Annatto Extract | 0.00 | 4.28 | 4.56 | 0.00 | 0.00 | 3.90 |
| Turmeric | 4.33 | 5.35 | 5.63 | 0.00 | 0.00 | 4.50 |
| Control | 6.95 | 6.95 | 6.95 | 6.48 | 6.48 | 6.48 |
| S. choleraesuis | | | | | | |
| Methylene blue (positive | 3.20 | 0.00 | 2.27 | 0.00 | 0.00 | 0.00 |
| control) | | | | | | |
| Hemin | 4.74 | 4.48 | 4.02 | 4.55 | 3.85 | 2.79 |
| Protoporphyrin | 2.92 | 3.67 | 3.40 | 3.32 | 2.33 | 2.10 |
| Annatto Extract | 0.00 | 2.27 | 2.86 | 0.92 | 0.00 | 1.63 |
| Turmeric | 3.92 | 3.82 | 2.81 | 3.47 | 1.73 | 2.37 |
| Control | 7.28 | 7.28 | 7.28 | 7.28 | 7.28 | 7.28 |

### Example 3

### Antifungal Activity

Test microorganisms included Aspergillus niger and Fusarium solani. The test organisms were grown in potato dextrose broth. An organism suspension was transferred into potato dextrose broth and incubated at 28° C. for 72 hours. The test Compound was Annatto extract and Turmeric and was prepared as 1, 0.1, and 0.01%, by weight, in 30% Mueller Hinton Broth in phosphate buffered saline.

Using a sterile 96-well microtitre plate under the laminar flow hood, the following was done to set up the above challenge plate for 1 organism per plate/per light exposure per time point. To a 96 well nunc plate 150 μl of the potato dextrose broth containing the 72 hour culture was added to each well. The MBEC plate lid was placed into the inoculum. The devices were put on an orbital shaker set at 110 RPM and Incubated at 28° C. for 48 hours.

The compounds were added to distilled water in the challenge plate. The planktonic microorganisms were rinsed off by dipping the lid into distilled water for 1-2 minutes. The lid was transferred to the challenge plate and let sit for 20 minutes at room temperature with a tin foil cover.

The pegs with biofilm were exposed to light sources (full spectrum fluorescent light or LED light) by placing the devices on a rack. Both light sources were exposed for 16 hours. The fluorescent exposure was done in a cold room with 2 light fixtures with two fluorescent bulbs each. The entire assembly was covered in tinfoil. The LED exposure was done by placing the light source above the plate on the rack and then placing a tinfoil wrapped box over the rack. Following the 16 hour exposures, rinse plates were prepared by adding 0.9% sterile saline (200 μL per well) to each well of a sterile 96 well micro titre plate. The peg lids were rinsed twice in 0.9% sterile saline for 1 to 2 minutes.

The pegs were transferred to PDP recovery media, and then the recovery plate was sealed in tin foil (or other opaque covering) and incubated at 28° C. for 24 to 48 hours.

The MFC was determined by incubating the planktonic plate at 28° C. for 96 hours and read visually to determine MBC values. The MBEC was determined by incubating the recovery plate at 28° C. for 96 hours, and read visually to confirm MBEC values.

Both biofilm and planktonic forms of Aspergillus and Fusarium were killed by Turmeric or Annatto in combination with LED or fluorescent lights (Table 3). The data suggest a difference between the light source and the activity of the photodynamic agent.

TABLE 3
The number of test sites sterilized by the Photodynamic activity
of Annatto extract or Turmeric with LED lights or Fluorescent Lights

| | MBEC | | | MBC | | |
|---|---|---|---|---|---|---|
| | 1% | 0.1% | 0.01% | 1% | 0.1% | 0.01% |
| | LED Light | | | | | |
| Aspergillus | | | | | | |
| Annatto | 3/3 | 3/3 | 3/3 | 3/3 | 3/3 | 3/3 |
| Extract | | | | | | |
| Turmeric | 3/3 | 3/3 | 3/3 | 3/3 | 3/3 | 3/3 |
| Fusarium | | | | | | |
| Annatto | 0/3 | 0/3 | 0/3 | 1/3 | 2/3 | 2/3 |
| Extract | | | | | | |
| Turmeric | 2/3 | 3/3 | 2/3 | 0/3 | 1/3 | 2/3 |
| | Fluorescent Light | | | | | |
| Aspergillus | | | | | | |
| Annatto Extract | 3/3 | 0/3 | 0/3 | 3/3 | 0/3 | 0/3 |
| Turmeric | 0/3 | 0/3 | 0/3 | 0/3 | 0/3 | 0/3 |
| Fusarium | | | | | | |
| Annatto Extract | 1/3 | 0/3 | 0/3 | 2/3 | 0/3 | 1/3 |
| Turmeric | 2/3 | 2/3 | 0/3 | 0/3 | 0/3 | 0/3 |

MFC = Minimum Fungicidal Concentration
MBEC = Minimum Biofilm Eradication Concentration
MBC = Minimum Biocidal Concentration

### Example 4

### Activity of Photodynamic Agent on Plant Bacterial Pathogens

Test microorganism included Pseudomonas syringae, a food spoilage microorganism and plant pathogen. Test Compounds included Hemin (blood product), Annatto Extract, and Turmeric. Compounds were prepared as 1%, 0.1%, 0.01%, by weight, in 30% Mueller Hinton Broth in phosphate buffered saline. The light source was a broad spectrum fluorescent light or an LED light and the contact time was 16 hours. The study temperature was 28° C.

Approximately 10⁶ organisms in 150 uL Cationic

Adjusted Mueller Hinton Broth were placed in each of the wells of a 96 peg MBEC device. The lid of the 96 peg MBEC device was placed on the bottom plate containing organism. The device was placed on a shaker, set to 110 rpm, and incubated at 28° C. for 24 hours.

The challenge plates were set up using a sterile 96-well microtitre plate using aseptic techniques in the following manner (one organism per plate, per light exposure time, per light source). Test solutions (200 uL) were placed into each well. The lid was transferred to the challenge plate and exposed to the various light sources for the prescribed exposure times (16 hours). Following the treatment, the pegs were transferred to the recovery media (“recovery plate”) then wrapped in tin foil and incubated at 28° C. for 18 to 24 hours.

The recovery plate(s) was wrapped in tin foil, incubated at 28° C. for 24 hours and read visually and by a plate reader, set to an optical density measurement of 630 nm, to determine MBEC growth.

Hemin, Annatto extract and Turmeric at 1%, 0.1% and 0.01% concentrations and 16 hours of LED or fluorescent exposure was an effective anti-microbial agent against P. syringae showing a reduction in growth (>90%) (Table 4).

TABLE 4
Average Log₁₀ growth reductions (log10 CFU) of Pseudomonas
syringae HB9 in 1%, 0.1% and 0.01% concentrations of various
photocatalytic agents when exposed to 16 hours of LED light. Samples
were run in triplicate.

| | LED Light | | | Fluorescent Light | | |
|---|---|---|---|---|---|---|
| Test Compound | 1% | 0.1% | 0.01% | 1% | 0.1% | 0.01% |
| Hemin | 3.97 | 1.32 | 1.48 | 1.51 | 0.92 | 1.07 |
| Annatto extract | 1.51 | 1.16 | 1.64 | 3.75 | 1.50 | 1.04 |
| Turmeric | 1.39 | 1.63 | 1.20 | 0.94 | 1.32 | 1.85 |

### Example 5

### Use of Photodynamic Agents for Sanitization of Hard Surfaces

Test microorganisms included Salmonella choleraesuis, E. coli 0157:H7, and Staphylococcus aureus. Test dye Compounds included Annatto Extract, and Turmeric. Compounds were prepared as 0.01%, by weight, in 30% Mueller Hinton Broth in phosphate buffered saline.

The light source was a broad spectrum Fluorescent light and the contact time was 16 hours. The study temperature was 4° C. The surfaces tested were wood, stainless steel, mild steel, rubber, silicone, laminated plastic, and ultra high molecular weight plastic. All materials are used in food processing operations. Equal sized samples of materials listed were prepared by mounting them onto the pegs in a 12-well BEST lid making sure that all samples formed extended approximately the same distance from the lid. The BEST lid and assay procedure is described in U.S. Pat. Nos. 7,041,470; 6,410,256; 6,326,190; 6,051,423; and 6,599,714, all incorporated herein by reference in their entirety.

An in-vitro assay system for evaluating adhesion, post adhesion killing and inhibition of biofilm formation on catheter surfaces using the BEST Assay. An inoculum in 3 mL sterile water matching a 0.5 McFarland Standard (1.5×10⁸ cells per mL) was created in a glass test tube using a sterile cotton swab. The 0.5 McFarland solution was diluted 88 μl in 22 mL 30% Mueller Hinton Broth in PBS and 4 mL of the inoculums was placed into each designated well of a 12-well plate.

The BEST device with the attached samples was placed into the filled 12-well plate. The BEST device was resealed and placed on a rotary shaker (110 revolutions per minute) and incubated at 37±1° C. for 18 to 24 hours.

All photodynamic reagents were prepared by weighing the specific amount of dye using a sterile spatula and adding it to sterile 30% Mueller Hinton Broth in PBS. Challenge plates were prepared by filling a 12-well plate with 4.5 mL of photodynamic agent, which completely immersed the test surfaces. After 18 to 24 hours of biofilm growth, the BEST devices were rinsed once in sterile water, then transferred to the challenge plate. The test surfaces with the bacterial biofilms were allowed to contact the agent for 5 minutes in darkness. The test surfaces were then exposed to the test light source for the test exposure time.

After exposure, the samples on the peg lid were rinsed 3 times in sterile water by dipping the lid with samples attached into 3 consecutive bottom plate devices containing 5 ml of sterile water in each well. The BEST lid was then inserted into a 12 well plate containing 4.5 ml of universal neutralizer. The entire device was sonicated for 30 minutes. Following sonication, 100 μl from each well of the BEST plate was placed into the first 12 empty wells of the first row of a 96 well microtiter plate. 180 μl of 0.9% sterile saline was placed in the remaining rows. A serial dilution (10°-10⁻⁷) was prepared by moving 20_l down each of the 8 rows. 20 μl from each well was removed and spot plated on a prepared Trypticase Soy Agar plate. Plates were incubated at 37±1° C. and counted after approximately 24 hours of incubation.

The results in Table 5 correspond to Log₁₀ reduction in number of viable bacterial biomass after exposure to photodynamic agents. This biomass was represented as cfu/mm² on each surface.

Both turmeric and annatto extract were able to cause a significant reduction in biofilm on the surface of these materials. There was a difference in the preference of photodynamic agent depending on the challenge microorganism and surface. For example, Turmeric performed best for E. coli and Salmonella; Annatto extract was best for Staphylococcus aureus.

This data suggests that a combination of Turmeric and Annatto would be preferred. Wood surface is one of the most difficult surfaces to sanitise. Its porosity provides a good environment for biofilm to grow away from light.

TABLE 5
Log₁₀ reduction values of various photocatalytic reagents
applied to E. coli 0157:H7 growing on stainless steel,
mild steel, UHMWP, laminated plastic, silicone, rubber,
wood, and concrete after a 16 hour exposure time.

| | | E. coli 0157:H7 | S. choleraesuis | S. aureus |
|---|---|---|---|---|
| Stainless | Annatto | 0.43 | 0.21 | 0.41 |
| steel | Turmeric | 2.08 | 2.27 | −0.03 |
| Mild steel | Annatto | 0.64 | -0.04 | −2.90 |
| | Turmeric | 0.79 | 2.09 | −3.15 |
| HMWP | Annatto | 1.79 | 0.66 | 1.71 |
| | Turmeric | 4.33 | 3.79 | 4.80 |
| Laminated | Annatto | 0.39 | −3.65 | 1.88 |
| Plastic | Turmeric | 1.20 | −3.00 | 1.76 |
| Silicone | Annatto | 1.24 | 0.50 | 1.25 |
| | Turmeric | 3.88 | 2.66 | 1.84 |
| Rubber | Annatto | 0.15 | 0.20 | 0.98 |
| | Turmeric | 0.29 | 0.19 | 0.63 |
| Wood | Annatto | −0.47 | −0.58 | 1.61 |
| | Turmeric | −0.44 | −0.36 | 0.30 |
| Concrete | Annatto | 0.56 | 0.29 | 1.65 |
| | Turmeric | 1.58 | 0.55 | 1.41 |

### Example 6

### Use of Photodynamic Agents for Sanitization of Foods

Test microorganisms included Salmonella choleraesuis, E. coli 0157:H7, Staphylococcus aureus, Campylobacter jejuni, and Listeria monocytogenes. The test Compound was Turmeric, prepared as 0.01%, by weight, in 30% Mueller Hinton Broth in phosphate buffered saline.

The light source was a broad spectrum Fluorescent light and the contact time was 16 hours. The study temperature was 4° C. The surfaces tested were beef and poultry. Using sterile shears the skin/surface of the meat products (Beef or poultry) was cut into pieces that fit into the bottom of a 9 cm Petri dish.

An 8 mm biopsy punch was used to take a sample of each piece of meat. The sample was used as the sterile control and to provide the background contamination count of the meat. 200 μl of a 0.5 McFarland standard, of an organism to be tested, was placed onto a single piece of meat in a Petri dish and spread using a sterile spreader. Inoculated samples were placed in an incubator for 1 hour incubation at Room temperature.

Following incubation, the piece of meat, were cut into 3 pieces (one for the positive control (no PDA applied) and two for the test compounds). One of the concentrations of one photodynamic dye was applied to one of the “Test compound section” of the meat using a spray bottle. A portion down the middle was left untreated and used as a growth control. This gave 2 dyes per piece with one common growth control.

The test device was exposed to the test light source for the test exposure time. Following the 16 hour light exposure an 8 mm biopsy punch was used to cut small sections out of the piece of meat. The piece of meat was homogenised in a 15 ml tube with 2 ml of sterile of CAMHB with 0.5% Tween 80. Following homogenisation the samples were serially diluted and spot plated on Trypticase Soy Agar plate or a Brain Heart Infusion Agar (For Listeria spp.). Plates were incubated at 37±1° C. and counted after approximately 24 hours of incubation. Data was evaluated as CFU/mm². The results show that 0.1% Turmeric caused a reduced colonization of Salmonella choleraesuis, E. coli 0157:H7, Staphylococcus aureus, Campylobacter jejuni, and Listeria monocytogenes on the surface of chicken and beef (Table 6).

The results also appear to indicate that rough surfaces, e.g., the beef, may protect the bacteria from the light source. Slaughter house beef, beef covered with a layer of fat (smooth surface), allows light to better contact photodynamic agent, and therefore the PDA gives better results.

TABLE 6
Log₁₀ Reduction of Bacterial Colonization on Food Surface
using Turmeric as the Photodynamic agent and Fluorescent lights.

| | | Listeria | Staph. | | Salmonella | Campylobacter |
|---|---|---|---|---|---|---|
| | | monocytogenes | aureus | E. coli | cholerasuis | jejuni |
| Chicken | Turmeric | 0.62 | 1.09 | 0.02 | 1.29 | 1.32 |
| | 0.1% | | | | | |
| | Turmeric | 0.44 | 0.87 | 0.53 | 0.64 | −0.06 |
| | 0.01% | | | | | |
| Beef | Turmeric | 0.06 | 0.22 | 0.70 | −0.04 | 0.15 |
| | 0.1% | | | | | |
| | Turmeric | 0.44 | 0.21 | 0.08 | 0.85 | 1.06 |
| | 0.01% | | | | | |

### Example 7

Test microorganisms included Staphylococcus aureus, Listeria monocytogenes, E. coli ATCC 25922 and Salmonella choleraesuis ATCC 10708 foodborne pathogens of humans. Test Compounds are shown in Table 7. Compounds were prepared as described in Example 1.

TABLE 7

| | | | | | Dilution step to | |
|---|---|---|---|---|---|---|
| | | | | Stock | get the start | |
| | | | Chemical | Solution | conc. Ppm in | |
| Code | V. Product Name | State | Content | (ppm) | 10% w/v CAMHB | |
| A. | annatto powder, type 17.5G | powder | norbixin | N/A | 0.0125 | g/5 mL |
| B. | annatto extract WSL | liquid | norbixin | 69000 | 725 | μL/5 mL |
| C. | natural annatto liquid “Y” | liquid | annatto | 234000 | 215 | μL/5 mL |
| D. | natural orange color | liquid | annatto | 30000 | 1670 | μL/5 mL |
| E. | natural egg shade liquid | liquid | Annatto/ | 88000 | 570 | μL/5 mL |
| | | | curcumin | | | |
| F. | turmeric oleoresin WSL | liquid | curcumin | 519000 | 95 | μL/5 mL |
| G. | aquaresin turmeric | paste | curcumin | 129000 | 390 | μL/5 mL |
| H. | natural yellow shade liquid “IT4” | liquid | curcumin | 20800 | 2400 | μL/5 mL |
| I. | natural yellow shade liquid | paste | curcumin | 43800 | 1140 | μL/5 mL |
| | “CNW” | | | | | |
| J. | natural yellow color I | liquid | curcumin | 484000 | 100 | μL/5 mL |
| K. | natural yellow color II | liquid | curcumin | 48400 | 1030 | μL/5 mL |

CAMBH = Cation Adjusted Mueller Hinton Broth

The dyes worked with both biofilm and planktonic bacteria, as shown in Tables 8 and 9.

TABLE 8

| S. aureus | Concentration that eliminated all Bacteria as a | | | | | | | | | | |
|---|---|---|---|---|---|---|---|---|---|---|---|
| ATCC | Biofilm (MBEC) or as a Planktonic organism (MBC) | | | | | | | | | | |
| 29213 | A | B | C | D | E | F | G | H | I | J | K |
| MBC | 2500 | 10000 | 9.8 | 10000 | 9.8 | 9.8 | 9.8 | 9.8 | 9.8 | 39.8 | 9.8 |
| MBEC | 2500 | 10000 | 10000 | 10000 | 10000 | 10000 | 19.5 | 1250 | 10000 | 156.2 | 10000 |

TABLE 9

| | Concentration that eliminated all Bacteria as a Biofilm | | | | | | | | | | |
|---|---|---|---|---|---|---|---|---|---|---|---|
| L. monocytogenes | (MBEC) or as a Planktonic organism (MBC) | | | | | | | | | | |
| ATCC 29213 | A | B | C | D | E | F | G | H | I | J | K |
| MBC | 2500 | 10000 | 10000 | 10000 | 10000 | 9.8 | 9.8 | 9.8 | 9.8 | 19.1 | 19.1 |
| MBEC | 2500 | 10000 | 10000 | 10000 | 10000 | 156 | 9.8 | 1250 | 156 | 19.1 | 19.1 |

TABLE 10

| E. coli | | | | | | | | | | | |
|---|---|---|---|---|---|---|---|---|---|---|---|
| ATCC | Compound cut-off point (ppm) Visual Cut-off | | | | | | | | | | |
| 25922 | A | B | C | D | E | F | G | H | I | J | K |
| MBC | >2500 | 10000 | 5000 | >10000 | 10000 | >10000 | 9.8 | 9.8 | 9.8 | >10000 | 9.8 |
| MBEC | >2500 | >10000 | >10000 | >10000 | >10000 | >10000 | 625 | 9.8 | 19.5 | >10000 | 312.5 |

TABLE 11

| Salmonella | | | | | | | | | | | |
|---|---|---|---|---|---|---|---|---|---|---|---|
| choleraesuis | Compound cut-off point (ppm) Visual Cut-off | | | | | | | | | | |
| ATCC 10708 | A | B | C | D | E | F | G | H | I | J | K |
| MBC | 2500 | 10000 | 10000 | >10000 | 10000 | >10000 | >10000 | 9.8 | 19.5 | >10000 | 39.1 |
| MBEC | 2500 | 10000 | 10000 | >10000 | 10000 | >10000 | 10000 | 9.8 | 39.1 | >10000 | >10000 |

### REFERENCES

Chan Y, Lai C—H (2003) Bactericidal effects of different laser wavelengths on periodontopathic germs in photodynamic therapy. Laser Med Sci 18: 51-55.
Abhay Sankar Chakraborti (2003) Interaction of porphyrins with heme proteins—a brief review. Molecular and Cellular Biochemistry 253: 49-54.
Margaret E. Daub and Marilyn Ehrenshaft (2000) The Photoactivated Cercospora Toxin Cercosporin: Contributions to Plant Disease and Fundamental Biology Annual Reviews of Phytopatholology. 2000. 38:461-90.
A. Egyed (1972) A Simple Method for Heme Isolation, Spcciaiia 15.11 1397-1398.
J. R. Guimarães and A. S. Barretto (2003) PHOTOCATALYTIC INACTIVATION OF Clostridium perfringens AND COLIPHAGES IN WATER Molecular and Cellular Biochemistry 253: 49-54.
Michael R. Hamblin, Jennifer Viveiros, Changming Yang, Atosa Ahmadi, Robert A. Ganz and M. Joshua Tolkoff (2005) Helicobacter pylori Accumulates Photoactive Porphyrins and Is Killed by Visible Light Antimicrobial Agents and Chemotherapy, 49: 2822-2827.
Giulio Joni and Stanley B. Brown (2004) Photosensitized inactivation of microorganisms Photochemistry and Photobiological Science 3: 403-405
A. Kubin, G. Alth, R. Jindra, G. Jessner, R. Ebermann, (1996) Wavelength-dependent photoresponse of biological and aqueous model systems using the photodynamic plant pigment hypedcin. Journal of Photochemistry and Photobiology B: Biology 36: 103-108.
Yeshayahu Nitzan, Bracha Shainberg, and Zvi Malik (1997) Photodynamic Effects of Deuteroporphyrin on Gram-positive Bacteria Current Microbiology 251-258.
Tony P. Paulino, Prislaine P. Magalhaes, Geraldo Thedei, Jr., Antonio C. Tedesco, and Pietro Ciancaglini (2005) Use of Visible Light-based Photodynamic Therapy to Bacterial Photoinactivation. Biochemistry and Molecular Biology Education 33: 46-49.
C. R. Rovaldi, A. Pievsky, N. A. Sole, P. M. Friden, D. M. Rothstein, And P. Spacciapoli (2000) Photoactive Porphyrin Derivative with Broad-Spectrum Activity against Oral Pathogens In Vitro Antimicrobial Agents and Chemotherapy 44: 3364-3367.
H. Wainwright, D. A. Phoenix, J. Marland, D. R. A. Wareing, F. J. Bolton (1997) A study of photobactericidal activity in the phenothiazinium series FEMS Immunology and Medical Microbiology 19: 7580

We claim:

1. A composition for the treatment infection or contamination comprising at least one photodynamic agent comprising a natural or synthetic food dye.

2. The composition of claim 1 wherein the food dye is selected from the group consisting of annatto extract, turmeric, porphyrin containing compositions, and combinations thereof.

3. The composition of claim 1 wherein the concentration of food dye is sufficient to inactivate one or more microorganisms when the food dye is activated by a source of light.

4. A method for treating infections and contaminants on surfaces comprising:
a) Administering photodynamic agents selected from a group consisting of natural or synthetic food dyes;
b) Activating the agent by exposing the agent to a source of light; and
c) Inactivating organism and microorganisms.

5. The method of claim 4 wherein administering comprises administering to a surface comprising hard surfaces, food surfaces, and living human, animal and plant tissue.

6. The method of claim 4 wherein the treatment of infections and contaminants comprises treating one or more organisms or microorganisms.

7. The method of claim 4 wherein the organisms and microorganisms are viral, bacterial, fungal, algal or parasites.
